(19) United States
(12) Patent Application Publication
(10) Pub. No.: US 2026/0311848 A1
(43) Pub. Date: Oct. 8, 2026

(54) REGION-BASED MOTION CORRECTION USING EXTRA MODAL INFORMATION FOR SINGLE PHOTON EMISSION COMPUTED TOMOGRAPHY

(71) Applicant: Siemens Medical Solutions USA, Inc., Malvern, PA (US)

(72) Inventors: Alexander Hans Vija, Evanston, IL (US); Maximilian Reymann, Erlangen (DE)

(21) Appl. No.: 19/481,210

(22) PCT Filed: Aug. 10, 2023
(86) PCT No.: PCT/US2023/071978

§ 371 Date: Nov. 4, 2025

(51) Int. Cl. G16H 30/40; G06T 7/20; G06T 12/20

(52) U.S. Cl. CPC G16H 30/40; G06T 7/20; G06T 12/20; G06T 2207/10108

(57) ABSTRACT

For correction of motion in single photon emission computed tomography (SPECT) imaging, extra modal information is used to delineate different regions in a patient. Motion in these different regions as reflected in SPECT data is determined. These regional motions are used for motion correction. Statistical measurements for the SPECT data of the different regions may be used to weight the regional motions.

### BACKGROUND

The present embodiments relate to motion correction in single photon emission computed tomography (SPECT) imaging. Patient motion (e.g., respiratory, voluntary, involuntary, or activity shifts) may cause blur and/or artifacts in SPECT imaging.

Analysis of the consistency of the SPECT data itself can enable the correction of motion in SPECT projection data. A one-dimensional (1D) respiratory motion signal may be used to correct for the motion of the entire projection data. However, respiratory motion is a 3D heterogeneous motion.

View-by-view inconsistencies in SPECT data due to motion may be corrected using extra-modal data. Respiratory motion may be corrected using SPECT data driven methods. These corrections do not manage multiple motions in a dataset and assume all objects within the frame move with the same trajectory. If there are multiple moving objects or features in a dataset that result in a constant center of light, these methods do not correct any motion.

### SUMMARY

By way of introduction, the preferred embodiments described below include methods, systems, and non-transitory computer readable media for correction of motion in SPECT imaging. Extra modal information is used to delineate different regions in a patient. Motion in these different regions as reflected in SPECT data is determined. These regional motions are used for motion correction. Statistical measurements for the SPECT data of the different regions may be used to weight the regional motions.

In a first aspect, a method is provided for correction of motion in a single photon emission computed tomography (SPECT) imaging system. A SPECT detector detects emissions over time from a patient. The detected emissions include first projection data and are subject to the motion of the patient. Regional motions are estimated for different regions of the patient from the first projection data. The different regions are designated by extra modal information. The first projection data or data derived from the first projection data is motion corrected based on the estimated region motions. A SPECT image from the motion corrected first projection data or the data derived from the first projection data is displayed.

As a further approach, the extra modal information is segmented, providing masks for the different regions. The different regions are for different anatomy or functional area of the patient. In a further approach, the extra modal information as segmented is projected, the projection generating the masks.

In one embodiment, the extra modal information is computed tomography, magnetic resonance, ultrasound, positron emission tomography, or x-ray imaging of the patient.

In another embodiment, the motion of the patient is respiratory motion of the patient. The motion correction is for the respiratory motion.

According to another embodiment, the regional motions are estimated as amplitude of the first projection data as a function of time for each of the different regions.

In yet another embodiment, a statistical measurement is determined for each of the different regions. The motion correction is based on the estimated region motions and the statistical measurements. For example, the statistical measurement is count rate density of the respective region, count rate density ratios between the different regions, maximum range of motions within the different regions, adjacent view motion from an adjacent view of the SPECT detector, noise, or signal-to-noise ratio. Other statistical measurements may be used. The motion correction is based on the estimated region motions weighted by the statistical measurement for each of the different regions.

As one embodiment, the motion correction is based on a weighted combination of the regional motions. The weighting in the weighted combination is based on a statistical measurement. For example, the weighting selects one of the region motions based on the statistical measurement.

In one embodiment, the motion correction corrects the first projection data as list mode data or a frame prior to reconstructing the SPECT image. In another embodiment, the motion correction corrects reconstruction data in a reconstruction. The reconstruction data is the data derived from the first projection data.

In a second aspect, a method is provided for correction of motion in a single photon emission computed tomography (SPECT) imaging system. A motion correction to counteract the motion is determined. The determination is based on region delineation from information of a different modality than the SPECT. A SPECT image as motion corrected using the motion correction is generated.

In one implementation, the region delineation includes segmentation of different organs. The motion correction is determined from a set of region motions for the different organs.

As another implementation, the different modality is computed tomography, magnetic resonance, ultrasound, positron emission tomography, or x-ray. The region delineation is segmentations forward projected to a projection data space of SPECT data. The motion correction is from different motions determined from the SPECT data using the segmentations as forward projected.

In yet another implementation, the motion correction is determined as a weighted combination of regional motions from the region delineation.

For example, the weighted combination weights the regional motions using a statistical measure of SPECT data of respective regions from the region delineation.

In a third aspect, a single photon computed tomography (SPECT) system is provided. A SPECT detector is provided for detecting signals from a patient. A motion processor is configured to determine motions for different regions of the patient from the detected signals. The different regions are segmented from imaging of a modality other than the SPECT. A reconstruction processor is configured to reconstruct a SPECT image of the signals from the patient. The SPECT image is motion corrected based on the motions for the different regions. A display is configured to display the SPECT image.

According to one approach, the reconstruction processor is configured to apply motion correction to the signals as frames or list mode data prior to the reconstruction of the SPECT image. The motion correction is a weighted combination of the motions of the different regions.

In another approach, the weighted combination has weights based on statistical measures of the detected signals.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for correction of motion in a SPECT system;

FIG. 2 illustrates a method for correction of motion in SPECT imaging;

FIG. 3 is a block diagram of a SPECT imaging system, according to one embodiment, for motion correction.

### DETAILED DESCRIPTION OF THE DRAWINGS AND PRESENTLY PREFERRED EMBODIMENTS

Region-specific motion correction is provided for SPECT using extra modal information. Motions in specific sub-areas of the SPECT projection data are estimated given the definition of regions in data space (e.g., through forward projection of extra modal data such as computed tomography (CT), magnetic resonance, deep learning-generated segmentations, or through masks defined by users using conventional or time of flight cameras). This allows the determination of one or more 1D or 2D motion estimates for each region that have the same or different motions (amplitudes/frequencies/phase). For example, forward projected CT segmentation of organs is used to obtain a respiratory surrogate for each individual region in the SPECT data space.

Regional and/or global statistics may be used to determine the robustness of each motion estimate. For example, the sub-areas in SPECT data space are also used to extract statistics from the data, such as count rate density, count rate density ratios between the areas, maximum range of motions within the regions, motion estimate from adjacent views, noise, or signal-to-noise ratio. A combination of the regional motion estimates incorporates the statistics, such as by allowing a physician, expert, or algorithm to determine the reliability of each motion estimate and the final amount of motion correction applied to the different regions. The final motion correction may be application specific and may be different for one dataset depending on the task at hand. The final motion correction might be locally different or globally consistent based on the current data.

Generating only one respiratory motion correction factor for the entire projection data may fail to account for a 3D heterogenous motion observed in the projection data. By combining region-based count analysis with localized motion extraction, an application specific and hence more precise motion correction can be performed. Where two or more objects in SPECT data space are moving such that the center of light remains constant, motion in individual regions is detected and may be better corrected using regional estimates of motion from extra modal data. The 2D heterogenous motion in the data may be corrected due to the regional motion analysis.

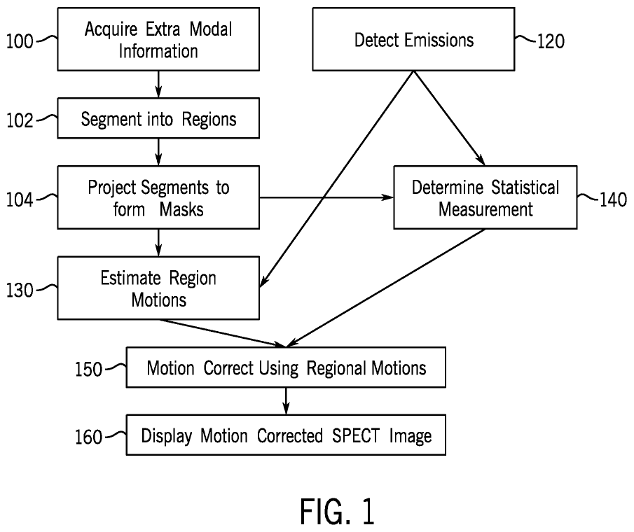

FIG. 1 shows one embodiment of a method for correction of motion in a SPECT imaging system. Motion from respiration or other sources is, at least in part, removed for the SPECT imaging. Regional analysis of the SPECT data allows for correction of motion even where the center of light (i.e., center of emissions or count locations) does not shift due to the motion. The motion correction to counteract the motion is determined based on region delineation from information of a different modality than the SPECT. SPECT data statistics by region may be used for selecting or combining the regional motions to determine a global motion.

The method is implemented by a SPECT imaging system, such as the SPECT system 300 of FIG. 3. An image processor segments in act 102, projects in act 104, estimates in act 130, and determines in act 140. The image processor or another imaging system acquires in act 100. A detector, such as a gamma camera, detects emissions in act 120. A display or printer may be used by the image processor for the display of act 160. Other devices may be used to perform any of the acts, such as a graphics processing unit and/or reconstruction processor.

The acts are performed in the order shown (e.g., top to bottom or numerical) or other orders. For example, acts 100-104 are performed prior to, simultaneously with, or after act 120. As another example, act 140 may be performed before, after, or simultaneously with any of acts 100, 102, 104, and/or 130.

Additional, different, or fewer acts may be performed. For example, act 140 is not provided where the motion correction is based on regional estimates without statistical weighting. As another example, act 160 may not be provided, such as where an image is later generated from motion corrected data and/or transmitted to memory or an electronic medical record. In other examples, acts for reconstruction are provided. Acts for configuring the imaging system or use of results may be provided. Acts for user input to segment, indicate preference for region, and/or control the motion correction may be provided.

In act 100, the image processor acquires extra modal information. The information is acquired from memory or transfer, such as from an imaging system. Scan or imaging data from a previous or con-current scan of the patient is acquired. Alternatively, an imaging system acquires the extra modal information by scanning the patient. For example, a CT system housed with or part of the SPECT imaging system acquires a CT representation of the patient.

The extra modal information is information other than SPECT data. Data representing the patient in a different modality than the detected SPECT emissions from the patient is acquired. For example, CT, magnetic resonance, ultrasound, positron emission tomography, or x-ray imaging of the patient is acquired. Any extra modal information that provides a segmentation may be used. For example, a Doseless MuMap estimate may be used. As another example, a camera image captures the outside of the patient, and a machine-learned model (artificial intelligence) generates a representation of the patient and/or segmentations of the interior of the patient. In yet another example, a statistical shape model is fit to the patient (e.g., fit to CT or camera image of the exterior) where the shape model as fit indicates locations of anatomy in the patient.

In act 102, the image processor segments the extra modal information. The spatial representation of the patient of the extra modal information is divided into different regions. For example, the segmentation is for different anatomy of the patient, such as identifying locations of the heart, gall bladder, and/or other organs. As another example, the segmentation is for different functional areas, such as a part of the lung with greater (e.g., high activity) uptake. The different anatomical structures or functional areas are segmented or delineated. In alternative embodiments, the user segments the extra modal information.

The segmentation in two or three dimensions. For example, the segmentation from CT or magnetic resonance imaging is three dimensional. The voxels representing the different anatomy or functional area are segmented from each other.

Any segmentation approach may be used. For example, pattern matching, random walker, thresholding, and/or filtering with region growing-shrinking-skeletonization may be used. In one approach, a machine-learned model or segmentation is applied. The extra modal information is input, and the machine-learned model outputs the locations of different anatomy or functional area. A user may segment.

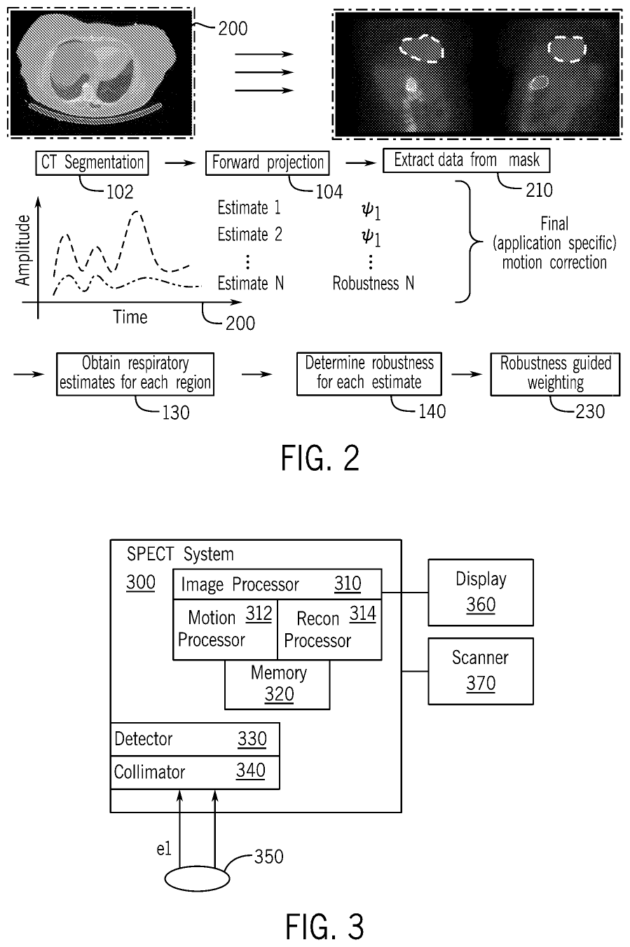

FIG. 2 shows an example segmentation. A CT slice 200 is shown. The CT slice 200 represents a plane through the patient. Multiple slices are provided for segmentation in three dimensions. The segmentation of act 102 is performed on the CT slice 200 and the other slices representing the volume of the patient.

In act 104, the image processor projects the segmentations. The segmentation provides masks for different regions in the extra modal space. Since SPECT acquires data (detects emissions) in a projection space (e.g., each detected emission is from along a line), the segmentations are forward projected to the projection space of the SPECT data. With the SPECT detector (e.g., gamma camera) at a given position relative to the patient (i.e., given view), the segmentations are projected to along the view direction. For different views, different forward projections are performed.

The extra modal information as segmented is generation of masks 210 (see dotted and dashed-line areas of FIG. 2) for different regions of the SPECT data. The region delineation in the SPECT data domain are segmentations forward projected to the projection data space of SPECT data. In the example of FIG. 2, the SPECT system includes two cameras, such as positioned to view the patient with a 90-degree difference between the two views. The segmentations are forward projected in act 104 for these two views. In each view, masks 210 are provided for different anatomy and/or functional area. For example, masks 210 for each view are provided for the heart, gall bladder, and/or other organs.

In act 120, a SPECT detector detects emissions over time from a patient. After ingesting or injecting a radiotracer into the patient, the patient is positioned relative to a detector, and/or the detector is positioned relative to the patient. Emissions from the radiotracer within the patient are detected over time. A collimator in front of the detector limits the direction of photons detected by the detector, so each detected emission is associated with an energy and line of response (e.g., a cone of possible locations from which the emission occurred). The detection along lines of response forms projection data, data representing emissions along lines generally perpendicular to the detector. The depth of the emission from the detector is not known.

For SPECT, the detector may be rotated or moved relative to the patient, allowing detection of emissions from different angles (views) and/or locations in the patient. At each position or view, the detector dwells for a period while detecting emissions. For multi-camera SPECT, emissions may be detected at a same time using cameras at different angles (views) relative to the patient.

The detector includes direct detection with CZT or indirect conversion (e.g., Nal, LSO layered scintillation crystal) using photomultiplier tubes, SiPM, or other photon detectors. For SPECT, detectors are arranged along a rectangular or other grid to provide a two-dimensional planar array for detecting gamma radiation along lines of response corresponding to the cells or pixels of the planar array. Other types of detectors may be used, such as any gamma detector.

The emissions over time are detected. Due to the detection along lines of response, the detections constitute projections of the emissions. By detecting at different locations (e.g., pixels in a SPECT detector), projection data (e.g., no depth or limited depth information) distributed over multiple dimensions is provided. For example, a SPECT detector at one location relative to the patient generates projection data distributed along two dimensions of the planar SPECT detector. The detector dwells at a given view to detect emissions over time, such as a dwell time of 10 seconds, 20 seconds, one minute, or more.

Since the detection of emissions takes seconds or minutes to gather sufficient data to avoid or limit artifacts due to poor signal-to-noise ratio (SNR), the detected emissions are subject to motion of the patient. As the patient is scanned, the patient breathes or moves. This motion causes parts of the patient to shift so that a detection from one location in the patient is at one pixel of the detector at one time, but another pixel of the detector at another time. The resulting projection data even with a same position of the detector relative to the patient is subject to blurring due to motion.

In act 130, the image processor estimates region motions for different regions of the patient. For each region (e.g., masked region or segmentation in projection space), motion is estimated from the projection data. The motions in the SPECT data for the different regions designated by extra modal information are separately estimated.

The motion is estimated in one or two dimensions. With a multi-camera SPECT system and the cameras (detectors) at different non-180-degree views, three-dimensional motion may be estimated. In one approach, the motion for each region is estimated as an amplitude of the projection data as a function of time. FIG. 2 shows the amplitude over time of the SPECT data for each of N regions, providing N estimates 220 of region motion. The motion may be estimated as frequency, phase, amplitude, direction, and/or combinations thereof.

The SPECT data for a given view is separated by temporal windows, providing gated SPECT data over time. The SPECT data for each time or temporal window is masked. Different masks result in each time or window having data separated by region. The masking removes data that is for other regions or anatomy.

The SPECT data may be processed in other ways before or after gating and/or masking. For example, spatial or temporal filtering is applied. As another example, scatter correction is applied.

For each time or temporal window, the intensities (e.g., count) for the locations in a given mask are summed, averaged, or otherwise combined, providing an amplitude of movement for the region. This amplitude over time provides a motion estimate 220 for the region. Estimates 220 are determined for different regions.

In an alternative approach, the image processor estimates the motion by tracking. The amplitude is of the motion vector or amount of change between times or temporal windows. The goal is to estimate rigid translations along the horizontal and vertical axes of the projection space.

Non-rigid translation may be used in other embodiments. Different translations and/or rotations between the SPECT data of different times are tested. The translation and/or rotation from a reference SPECT data distribution of one time with a best match of the SPECT data distribution of another time indicates the motion. Rather than pairwise estimation of motion, joint estimation may be used. The two-dimensional motion vectors are jointly estimated across the masked sets of SPECT emission data. The rigid motion is parameterized as horizontal and vertical or along other perpendicular axes. Other motion parameters than two-dimensional motion vectors may be estimated, such as scale and/or rotation.

The motion estimates 220 by region for the different regions are determined for each SPECT camera stop (view). For a dual-head SPECT system, a joint solution may include solving with two simultaneously acquired projections. Data from all or many projections are used in the solution or objective function.

Other motion estimation techniques may be used. For example, a statistical model is fit to the SPECT data over time for a particular region. Different models are provided for different anatomy. By fitting the model to the SPECT data, the parameters of the motion are determined.

In act 140, the image processor determines a statistical measurement for each of the different regions. The statistical measurement represents a quality or robustness of the SPECT data at that location. The statistical measurement represents a level of accuracy of motion for that region. The measurement may be an absolute value or may be a relative value (e.g., accuracy of motion of one region relative another region). By identifying regions with better quality or motion accuracy, a more correct motion or motions may be determined.

The statistical measurement is a count rate density of the respective region, count rate density ratios between different regions, maximum range of motion within the respective region, adjacent view motion from an adjacent view of the SPECT detector (e.g., amplitude of motion from an adjacent view (e.g., 2 degree angle) for the same region), noise, or signal-to-noise ratio. Other statistical measurements may be used.

The measurement is of the SPECT data. For example, the count rate density of the SPECT data for a given region (e.g., gall bladder) is calculated. A value of the statistical measurement is determined for each region. For each estimate 220 of motion, a robustness (statistical measurement) ψ is determined.

In act 150, the image processor motion corrects the projection data or data derived from the projection data. For example, the correction counters or accounts for respiratory and/or any other motion. An inverse of the motion is applied to shift the data to a motion corrected location.

The correction may be applied to the projection data. The motion correction is to the projection data as list mode data or a frame of SPECT data prior to performing reconstruction of the SPECT image. In an alternative or additional approach, the motion is used with the system matrix or projection operator to account for the motion for or during reconstruction.

The motion correction is applied to reconstruction data in the reconstruction. The reconstruction data is data derived from the projection data, such as in object or image space. The motion is used in the reconstruction, applying the motion in image space rather than SPECT data space. The reconstruction accounts for the motion while preserving the raw projection data and noise structure.

The correction is over time. Different amounts of motion are provided at different times, so the correction is different at different times or temporal windows. The estimated motion as a function of time is used to correct. The motion at each time represents a spatial offset. Each emission is detected as being for a line of response at a particular time. An inverse of the motion at that time is applied to the emission(s), shifting the location or line of response at which the emission(s) are detected. This correction process is applied to each detected emission or count.

The motion correction is a global correction. The estimated motions 220 of the different regions are combined to provide a global correction. The motion correction is determined from a set of region motions 220 for the different organs or other anatomy. The motions 220 of the SPECT data are combined to provide a motion for the SPECT data at that time or temporal window. The regional delineation is used to find separate region motions 220, which are then combined into a global motion to be corrected or undone.

The combination may be a selection, such as selecting the region motion of the organ of interest or better statistical measurement. A weighted combination of the region motions 220 may be used. For selection, one weight is 1 and the others are 0. For other weighted combinations, different weights are provided for the motions of different regions. After weighting, the weighted motions are summed or averaged.

FIG. 2 shows an example using a sum, resulting in a global motion 230 weighted by the statistical measurements. The motion correction is based on the estimated region motions 220 and the statistical measurements for those regions. The region delineation from the extra modal information is used for both region motion 220 and corresponding weighting. The statistical measurement for each region weights the region motion 220 for that region prior to combination. Other information may be used in weighting, such as application (e.g., organ of interest), user input, or user preference. The weighted combination of motion estimates may be based on reliability, local statistics, and/or global statistics

In an alternative approach, regional corrections are used. The SPECT data is corrected by region. Different regions may have different corrections due to the different or regional motions 220. A non-rigid transform is applied for motion correction.

In act 160, a display displays a SPECT image. The SPECT image is generated from the motion corrected projection data or from data derived from the projection data. The motion corrected list mode data or frames are reconstructed to generate the SPECT image with less motion artifact. An image object is reconstructed from the corrected projection data. The projection data for each camera position is corrected and then used together for reconstruction.

A reconstruction processor reconstructs an image object from acquired, motion corrected projection data from different camera angles. Computed tomography implements reconstruction to determine a spatial distribution of emissions from the detected lines of response. The position of each emission relative to the lines of response is corrected to account for patient motion. The corrected projection data represents the detected emissions and the lines of response for those emissions. The quantity or amount of uptake for each location (e.g., voxel) may be estimated as part of the reconstruction. The SPECT imaging system may estimate the activity concentration of an injected radiopharmaceutical or tracer for the different locations.

Alternatively, the motion correction is applied to reconstruction data derived from the projection data. The motion correction is applied in the reconstruction. The resulting SPECT image from either motion correction prior to or during reconstruction has less motion artifact. Where the center of light does not shift but motion occurs, the region motion-based motion correction may still reduce motion and corresponding artifact in the SPECT image. The SPECT image, as motion corrected using motion correction from motions of multiple regions, is generated by the image processor and displayed.

Any now known or later developed reconstruction methods may be used, such as based on Maximum Likelihood Expectation Maximization (ML-EM), Ordered Subset Expectation Maximization (OSEM), penalized weighted least squares (PWLS), Maximum A Posteriori (MAP), multi-modal reconstruction, non-negative least squares (NNLS), or another approach. Different types of reconstruction have different strengths and weaknesses.

The reconstruction provides a three-dimensional representation of the image object. The 3D spatial distribution of emissions is determined. To generate the image, the reconstructed emissions along a plane, slab, or volume are used. The image of the reconstructed image object is generated. The image of the patient or part of the patient is generated from the reconstruction. For qualitative SPECT, this distribution is used to generate an image. For quantitative SPECT, the activity concentration for each location (e.g., voxel) is determined. The reconstruction provides voxel values representing activity concentration. In one embodiment, data for one or more (e.g., multi-planar reconstruction) planes is extracted (e.g., selected and/or interpolated) from a volume or voxels and used to generate a two-dimensional image or images. Alternatively, the reconstruction is to a two-dimensional image plane.

The output may be a transmission. The transmission is of the SPECT image to a display. The transmission may be to a memory through a memory interface and/or to a patient medical record, server, or other computer connected through a network interface.

The SPECT image is of the patient. The image is a SPECT image showing distribution of activity in the patient. The image is of a two-dimensional or planar slice in the patient. Alternatively, the image is a rendering from volume data to the two-dimensional display, such as using ray casting, path tracing, surface rendering, or other three-dimensional rendering.

FIG. 3 shows a SPECT system 300. The SPECT system 300 detects emissions due to radioactive decay in a patient. The SPECT system 300 may provide qualitative or quantitative imaging.

The system 300 implements the method of FIG. 1, the method of FIG. 2, or another method. By using motion correction based on motion from different regions determined from extra modal information, the imaging may have less motion artifact or blurring, at least at the organ or organs of interest. The estimated motions are used for motion correction in reconstruction or for motion correction of projection data prior to reconstruction.

The SPECT system 300 includes an image processor 310, a memory 320, and a display 360. The image processor 310, memory 320, and/or display 360 are part of the imaging system with the detector 330 or are separate (e.g., a computer, server, or workstation). Additional, different, or fewer components may be provided. For example, the system 300 is a computer without the detector 330 and collimator 340. As another example, user input, patient bed, CT scanner 370, or other devices are provided. Other parts of the system 300 may include power supplies, communications systems, and user interface systems. The image processor 310 is formed from a motion processor 312 and a reconstruction processor 314. The motion processor 312 and reconstruction processor 314 are separate processors or may be formed from the same processor or hardware device.

The detector 330 is a gamma camera connected with a gantry. The gamma camera is a planar photon detector, such as having crystals or scintillators with photomultiplier tubes, SiPM, or another optical detector for detection signals e1 from the patient. Any now known or later developed gamma camera may be used. The gantry rotates the gamma camera about the patient. Other structures of detectors may be used. Multiple gamma cameras may be used, such as for detecting emissions from different views at a same time. Other components may be provided, such as the collimator 340.

The SPECT system 300, using the detector 330, detects emissions from the patient 350 for measuring uptake or physiological function. During scanning of a patient, the detector 330 detects emission events. The emissions occur from any location in a finite source (i.e., the patient). The radiotracer in the patient migrates to, connects with, or otherwise concentrates at specific types of tissue or locations associated with specific biochemical reactions. Thus, a greater number of emissions occur from locations of that type of tissue or reaction. With a SPECT detector 330, the emission events are detected at different positions and/or angles relative to the patient, forming lines of response for the events. The patient bed may move to define a field of view relative to the patient.

Due to motion, the location of the lines of response and resulting pixel or sensor of the detector 330 that detects the emission from the same location may be different at different times. The projection data of the detected emissions is subjected to a motion artifact due to respiratory motion.

The motion estimation processor 312 and the reconstruction processor 314 are separate hardware devices or may be one hardware device configured for different operations. The processors 312, 314 are of a same or different type of device. A general processor, digital signal processor, imaging processor, graphics processing unit, artificial intelligence processor, graphics processor, application specific integrated circuit, field programmable gate array, digital circuit, analog circuit, combinations thereof, or other now known or later developed device may be used. Each of the processors 312, 314 is a single device, a plurality of devices, or a network. For more than one device, parallel or sequential division of processing may be used. Different devices making up one of the processors 312, 314 may perform different functions, such as one processor (e.g., application specific integrated circuit or field programmable gate array) for reconstructing the object and another (e.g., graphics processing unit) for rendering an image from the reconstructed image object. In one embodiment, one or both processors 312, 314 are a control processor or another processor of SPECT system 300. In other embodiments, one or both processors 312, 314 are part of a separate workstation or computer. The hardware processors 312, 314 are configured by software, firmware, and/or hardware and operate pursuant to stored instructions to perform various acts described herein.

The motion estimation processor 312 is configured to perform acts 100, 102, 104, 130, 140, and/or 150 of FIG. 1 but may perform additional, different, or fewer acts. The motion estimation processor 312 is configured to determine motions for different regions of the patient from the detected signals. The different regions are delineated based on masks or segmentation from imaging of a modality other than the SPECT. For example, a CT representation of the patient 350 from the CT scanner 370 is used to generate masks in projection space for different anatomy or functional areas, providing different regions for estimating motion. The motion estimation processor 312 then estimates motions for different regions from the detected emissions. The motion estimation processor 312 may form a global motion from the motions of the different regions. The global motion may be a weighted combination where the weights are based on statistical information from the detected emissions.

The reconstruction processor 314 is configured to perform act 160 of FIG. 1 but may perform additional, different, or fewer acts (e.g., perform the motion correction in act 150). The reconstruction processor 314 is configured to reconstruct a SPECT image of the signals from the patient.

The detected emissions are reconstructed into an object or image space, from which an image for display on the display 360 is rendered or otherwise generated. This SPECT image is motion corrected based on the motions for the different regions. The SPECT image is motion corrected in reconstruction to the object or image domain or by correction of the projected data used to reconstruct.

The motion estimation processor 312, reconstruction processor 314, or another processor applies the motion correction. The regional motions estimated by the motion estimation processor 312 are combined to provide one rigid or non-rigid transform over time. The combination may be a weighted summation or average. The weights may be between 0 and 1 for each region. The weights may be selected based on various criteria, such as statistical measures, imaging application, diagnosis, user preference, user input, or other information. The weights may be binary, such as one weight being 1 and the rest being 0, for selection of one regional motion to use as the global motion.

The transform to reverse or counter the motion is applied to the projection data as list mode data or frames of data prior to reconstruction of the SPECT image. Alternatively, the transform is applied or used in reconstruction, such as applied to image or object domain data created in reconstruction. In another alternative, the motion correction is applied to the SPECT image or object after reconstruction.

The reconstruction processor 314 is configured to reconstruct a volume or object from emissions. Any reconstruction may be used to estimate the activity concentration or distribution of the tracer in the patient.

Forward and backward projection are used iteratively until a merit function indicates completion of the reconstruction (i.e., a best or sufficient match of the image object to the detected emissions).

The reconstruction processor 314 generates one or more images based on the reconstruction. Any given image represents the emissions from the patient. The image shows the spatial distribution, such as with a multi-planar reconstruction or a volume rendering. For quantitative imaging, the image represents accurate measures (e.g., in Bq/ml) of the activity concentration. Alternatively, or additionally, the image shows a quantity or quantities (e.g., alphanumeric) representing the activity concentration or specific uptake values for one or more locations or regions.

The display 360 is a CRT, LCD, plasma screen, projector, printer, or another output device for showing an image. The display 360 is configured by a display plane or buffer to display a SPECT image of the reconstructed functional volume of the patient.

The memory 320 is a buffer, cache, RAM, removable media, hard drive, magnetic, optical, database, or other now known or later developed memory. The memory 320 is a single device or group of two or more devices. The memory 320 is part of the SPECT system 300 or a remote workstation or database, such as a PACS memory.

The detected emissions, counts, locations, projected data frames, list mode data, extra modal information, global motion, region motion, masks, segments, region delineation, statistical measurements, and/or other information are stored in the memory 320. The memory 320 may store data at different stages of processing. The data is stored in any format.

The memory 320 is additionally or alternatively a non-transitory computer readable storage medium with processing instructions. The memory 320 stores data representing instructions executable by the programmed processors 310, 312 and/or 314. The instructions for implementing the processes, methods, and/or techniques discussed herein are provided on non-transitory computer-readable storage media or memories, such as a cache, buffer, RAM, removable media, hard drive, or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The following is a list of non-limiting illustrative embodiments disclosed herein:

Illustrative embodiment 1. A method for correction of motion in a single photon emission computed tomography (SPECT) imaging system, the method comprising: detecting, with a SPECT detector, emissions over time from a patient, the detected emissions comprising first projection data and are subject to the motion of the patient; estimating region motions for different regions of the patient from the first projection data, the different regions designated by extra modal information; motion correcting the first projection data or data derived from the first projection data based on the estimated region motions; and displaying a SPECT image from the motion corrected first projection data or the data derived from the first projection data.

Illustrative embodiment 2. The method of illustrative embodiment 1 further comprising segmenting the extra modal information, the segmenting providing masks for the different regions, the different regions comprising different anatomy or functional area of the patient.

Illustrative embodiment 3. The method of claim 2 further comprising projecting the extra modal information as segmented, the projection generating the masks.

Illustrative embodiment 4. The method of any one of illustrative embodiments 1-3 wherein the extra modal information comprises computed tomography, magnetic resonance, ultrasound, positron emission tomography, or x-ray imaging of the patient.

Illustrative embodiment 5. The method of any one of illustrative embodiments 1-4 wherein the motion of the patient comprises respiratory motion of the patient, and wherein motion correcting comprises motion correcting for the respiratory motion.

Illustrative embodiment 6. The method of any one of illustrative embodiments 1-5 wherein estimating comprises estimating amplitude of the first projection data as a function of time for each of the different regions as the region motions.

Illustrative embodiment 7. The method of any one of illustrative embodiments 1-6 further comprising determining a statistical measurement for each of the different regions, wherein motion correcting comprises motion correcting based on the estimated region motions and the statistical measurements.

Illustrative embodiment 8. The method of illustrative embodiment 7 wherein the statistical measurement comprises count rate density of the respective region, count rate density ratios between the different regions, maximum range of motions within the different regions, adjacent view motion from an adjacent view of the SPECT detector, noise, or signal-to-noise ratio, and wherein motion correcting comprises motion correcting based on the estimated region motions weighted by the statistical measurement for each of the different regions.

Illustrative embodiment 9. The method of any one of illustrative embodiments 1-8 wherein motion correcting comprises motion correcting based on a weighted combination of the region motions, weighting in the weighted combination being based on a statistical measurement.

Illustrative embodiment 10. The method of illustrative embodiment 9 wherein the weighting comprises selecting one of the region motions based on the statistical measurement.

Illustrative embodiment 11. The method of any one of illustrative embodiments 1-10 wherein motion correcting comprises motion correcting the first projection data as list mode data or a frame prior to reconstructing the SPECT image.

Illustrative embodiment 12. The method of any one of illustrative embodiments 1-11 wherein motion correcting comprises motion correcting reconstruction data in a reconstruction, the reconstruction data comprising the data derived from the first projection data.

Illustrative embodiment 13. A method for correction of motion in a single photon emission computed tomography (SPECT) imaging system, the method comprising: determining a motion correction to counteract the motion, the determination being based on region delineation from information of a different modality than the SPECT; and generating a SPECT image as motion corrected using the motion correction.

Illustrative embodiment 14. The method of illustrative embodiment 13 wherein the region delineation comprises segmentation of different organs, the motion correction determined from a set of region motions for the different organs.

Illustrative embodiment 15. The method of any one of illustrative embodiments 13-14 wherein the different modality comprises computed tomography, magnetic resonance, ultrasound, positron emission tomography, or x-ray, the region delineation being segmentations forward projected to a projection data space of SPECT data, the motion correction being from different motions determined from the SPECT data using the segmentations as forward projected.

Illustrative embodiment 16. The method of any one of illustrative embodiments 13-15 wherein determining the motion correction comprises a weighted combination of regional motions from the region delineation.

Illustrative embodiment 17. The method of illustrative embodiment 16 wherein the weighted combination comprises weighting the regional motions using a statistical measure of SPECT data of respective regions from the region delineation.

Illustrative embodiment 18. A single photon computed tomography (SPECT) system comprising: a SPECT detector for detecting signals from a patient; a motion processor configured to determine motions for different regions of the patient from the detected signals, the different regions segmented from imaging of a modality other than the SPECT; a reconstruction processor configured to reconstruct a SPECT image of the signals from the patient, the SPECT image being motion corrected based on the motions for the different regions; and a display configured to display the SPECT image.

Illustrative embodiment 19. The SPECT system of illustrative embodiment 18 wherein the reconstruction processor is configured to apply motion correction to the signals as frames or list mode data prior to the reconstruction of the SPECT image, the motion correction comprising a weighted combination of the motions of the different regions.

Illustrative embodiment 20. The SPECT system of illustrative embodiment 19 wherein the weighted combination has weights based on statistical measures of the detected signals.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention. Independent of the grammatical term usage, individuals with male, female or other gender identities are included within the term. I (WE) CLAIM:

1. A method for correction of motion in a single photon emission computed tomography (SPECT) imaging system, the method comprising:
detecting, with a SPECT detector, emissions over time from a patient, the detected emissions comprising first projection data and are subject to the motion of the patient;
estimating region motions for different regions of the patient from the first projection data, the different regions designated by extra modal information;
motion correcting the first projection data or data derived from the first projection data based on the estimated region motions; and
displaying a SPECT image from the motion corrected first projection data or the data derived from the first projection data.

2. The method of claim 1 further comprising segmenting the extra modal information, the segmenting providing masks for the different regions, the different regions comprising different anatomy or functional area of the patient.

3. The method of claim 2 further comprising projecting the extra modal information as segmented, the projection generating the masks.

4. The method of claim 1 wherein the extra modal information comprises computed tomography, magnetic resonance, ultrasound, positron emission tomography, or x-ray imaging of the patient.

5. The method of claim 1 wherein the motion of the patient comprises respiratory motion of the patient, and wherein motion correcting comprises motion correcting for the respiratory motion.

6. The method of claim 1 wherein estimating comprises estimating amplitude of the first projection data as a function of time for each of the different regions as the region motions.

7. The method of claim 1 further comprising determining a statistical measurement for each of the different regions, wherein motion correcting comprises motion correcting based on the estimated region motions and the statistical measurements.

8. The method of claim 7 wherein the statistical measurement comprises count rate density of the respective region, count rate density ratios between the different regions, maximum range of motions within the different regions, adjacent view motion from an adjacent view of the SPECT detector, noise, or signal-to-noise ratio, and wherein motion correcting comprises motion correcting based on the estimated region motions weighted by the statistical measurement for each of the different regions.

9. The method of claim 1 wherein motion correcting comprises motion correcting based on a weighted combination of the region motions, weighting in the weighted combination being based on a statistical measurement.

10. The method of claim 9 wherein the weighting comprises selecting one of the region motions based on the statistical measurement.

11. The method of claim 1 wherein motion correcting comprises motion correcting the first projection data as list mode data or a frame prior to reconstructing the SPECT image.

12. The method of claim 1 wherein motion correcting comprises motion correcting reconstruction data in a reconstruction, the reconstruction data comprising the data derived from the first projection data.

13. A method for correction of motion in a single photon emission computed tomography (SPECT) imaging system, the method comprising:
determining a motion correction to counteract the motion, the determination being based on region delineation from information of a different modality than the SPECT; and
generating a SPECT image as motion corrected using the motion correction.

14. The method of claim 13 wherein the region delineation comprises segmentation of different organs, the motion correction determined from a set of region motions for the different organs.

15. The method of claim 13 wherein the different modality comprises computed tomography, magnetic resonance, ultrasound, positron emission tomography, or x-ray, the region delineation being segmentations forward projected to a projection data space of SPECT data, the motion correction being from different motions determined from the SPECT data using the segmentations as forward projected.

16. The method of claim 13 wherein determining the motion correction comprises a weighted combination of regional motions from the region delineation.

17. The method of claim 16 wherein the weighted combination comprises weighting the regional motions using a statistical measure of SPECT data of respective regions from the region delineation.

18. A single photon computed tomography (SPECT) system comprising:
a SPECT detector for detecting signals from a patient;
a motion processor configured to determine motions for different regions of the patient from the detected signals, the different regions segmented from imaging of a modality other than the SPECT;
a reconstruction processor configured to reconstruct a SPECT image of the signals from the patient, the SPECT image being motion corrected based on the motions for the different regions; and
a display configured to display the SPECT image.

19. The SPECT system of claim 18 wherein the reconstruction processor is configured to apply motion correction to the signals as frames or list mode data prior to the reconstruction of the SPECT image, the motion correction comprising a weighted combination of the motions of the different regions.

20. The SPECT system of claim 19 wherein the weighted combination has weights based on statistical measures of the detected signals.
